(19) United States
(12) Patent Application Publication
(10) Pub. No.: US 2017/0128353 A1
(43) Pub. Date: May 11, 2017

(54) Tattoo Enhancement and Moisturizing Towelette Wipe

(71) Applicant: Body Wipe Company, Mundelein, IL (US)

(72) Inventors: James Bahcall, Lincolnshire, IL (US); Joel Saban, Mundelein, IL (US)

(21) Appl. No.: 15/343,614

(22) Filed: Nov. 4, 2016

Related U.S. Application Data
(60) Provisional application No. 62/252,857, filed on Nov. 9, 2015.

(51) Int. Cl. A61K 8/92; A61K 8/81; A61Q 1/00; A61K 8/02

(52) U.S. Cl. CPC A61K 8/92; A61K 8/0208; A61K 8/8141; A61Q 1/00; A61K 2800/54; A61K 2800/56

(57) ABSTRACT

A tattoo-enhancing wipe provides a wipe impregnated with two enhancing oils. The oils may be compounded with an emulsifier and water for improved dispersion and absorption.

### CROSS REFERENCE TO RELATED APPLICATION

This application claims the benefit of U.S. Provisional Application 62/252,857 filed Nov. 9, 2015, and is hereby incorporated by reference.

### BACKGROUND OF THE INVENTION

Tattoos are an application of ink onto and/or within the layers of the skin of a body, such as the epidermis or (learns layer. Tattoos are not a modern trend, as tattoos have been discovered on mummified human remains dating as far back as the Neolithic era.

Despite the permanent nature of tattoos, it is known that tattoos fade and once vivid colors may dull and lose their initial sheen. Tattoos may fade from a result of exposure to ultra-violet light, exposure to chlorine, and/or exposure to other chemicals.

A common method currently in use for delaying color fade in tattoos is to apply a copious amount of sunscreen to the surface of the tattoo to prevent tanning of the epidermis, or outer layer of the skin. The application of sunscreen works only to delay the fade of tattoo color and does not revitalize the tattoo. Tattoo creams are used to accelerate the healing rate of a recently applied tattoo and are not designed for enhancement of older, already healed tattoos. As such, they are not suitable for the enhancement of the optical characteristics of a tattoo.

### SUMMARY OF THE INVENTION

The present invention provides a tattoo enhancing oil in a convenient wipe form. The oil may be emulsified with water for improved application to the skin and may include a humectant and superabsorbent polymer to retain moisture.

It is thus a feature of at least one embodiment of the invention to provide a convenient mechanism for enhancing the color and definition of tattoos.

These particular objects and advantages may apply to only some embodiments falling within the claims and thus do not define the scope of the invention.

### BRIEF DESCRIPTION OF THE DRAWINGS

FIG. 1 shows an exemplary embodiment of a single-use, tattoo-enhancing wipe in a single-use package,

### DETAILED DESCRIPTION OF THE PREFERRED EMBODIMENT

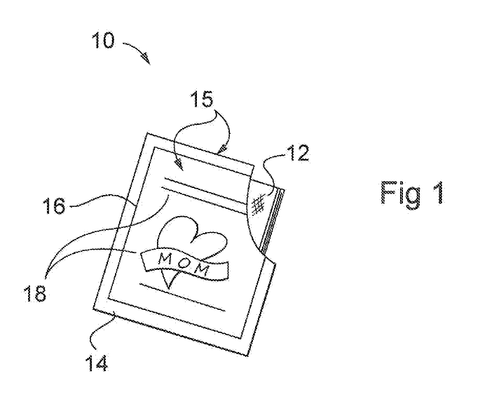

Referring now to FIG. 1 a single use tattoo-enhancing wipe package 10 may include a towelette 12 sealed in a tear, package 14 (shown with one corner removed to reveal the towelette 12). The tear package 14 has a front and rear plastic sheet 15 sealed around the periphery 16 to contain a square folded towelette 12 therein. The folded towelette 12 may be a woven or nonwoven absorptive fabric. The plastic sheets 15 may be printed on their outside with identifying information 18, and the front and rear plastic sheets 15 may include a light block such as foil or paper to shield the material of the towelette from both light and humidity.

The towelette 12 is impregnated with a tattoo-enhancing oil such as white mineral oil which may be emulsified, for example, by polyglyceryl-4 laurate to provide a lotion-like consistency for improved dispersion over the skin and moisturizing of the skin. While the applicant does not wish to be bound to a particular theory, it is believed that the oil and water improve the color transparency of the skin to enhance the color and definition of the underlying tattoo pigment. A humectant such as glyceryl and a superabsorbent polymer such as sodium, polyacrylate may he added to the towelette 12 to, preserve moisturizing effect.

In one embodiment the formulation impregnating the towelette 12 may be compounded by the following materials listed in order of weight:

TABLE I
Aqua
White mineral oil
Glycerol
Polyglyceryl-4 laurate/sebacate (and) polyglyceryl-4 caprylate/caprate
(and) water
Phenoxyethanol ipbc
Sodium polyacrylate
Squalane
Aloe barbadensis leaf juice
Benzenesulfonic acid, 2,2′-([1,1′-biphenyl]-4,4′-diyldi-2,1-ethenediyl) bis-,
sodium salt
Panthenol
dl-alpha-tocopheryl acetate
Isosqualane
Cold pressed, biodegradable oils
C-30 hydrocarbons

The combination of elements disclosed consists of multiple elements comprising a solution. The elements are a combination oft water, mineral oil, vegetable oil, a humectant, an emulsifier, a thickening agent, a moisturizer, aloe, a high-alkaline hydrocarbon, and a processing aid. Optionally, the solution may further contain any of the following: a vitamin E supplement, a phenoxyethanol IPBC, a sunscreen, a melanocyte inhibitor, an exfoliator, an optical brightening agent, and/or a fluorescent whitening agent. The combination may be an assortment of these elements, and does not need to be all of the elements to embody the present concept.

The composition disclosed herein contains a variety of elements, one of which is water. There are different types of water, such as distilled, de-ionized, filtered water, and the like, but any type of water will do and is not distinguished with regard to the present disclosure.

The composition disclosed in the present application is known to contain an amount of mineral oil. Mineral, oil, as defined herein, is a term referring to a variety of similar, yet different mixtures of alkalies and/or cycloalkanes. Mineral oil, as contemplated herein, may be originating from a non-vegetable mineral source, or may be obtained from crude petroleum or crude oil. Typically, mineral oil is colorless and odorless, though it may be later perfumed, with a carbon number range between about or around 15 and about or around 50 and may be composed mainly of alkanes and cycloalkanes, related to petroleum jelly. Additionally, mineral oil is known to have a density around 0.8 g/cm³, though it is contemplated it could go above or below this number slightly, but preferably is below 0.9 g/cm³. Common synonyms for the mineral oil used as in the present application are: base oils, mineral base oils, lubricant base oils, liquid paraffin, white oil, liquid petroleum, baby oil, naphthenic oil, aromatic oil, paraffinum liquidum, or paraffinum subliquidum. The mineral oil may be any one of the preceding types, but preferably it is white mineral oil.

Glycerol, as is well known, is used generally as a humectant while providing smoothness and lubrication. As used herein, a humectant refers to a material that promotes the retention of moisture. In a preferred embodiment, glycerol is used as a humectant in the disclosed solution, but alternative embodiments utilize substitute humectants, such as: glycol, aloe vera gel, sorbitol, dicyanamide, sodium lactate, honey, egg yolk, egg white, sugar alcohols, lithium chloride, neoagarobiose, urea, or quillaia. When the humectant is added to the combination, the solution created gains the ability to moisturize the skin and expedites the process of shedding, dry skin of the subject used. It was found that compositions containing shea butter have a density of around 0.9 g/cm³. By not adding shea butter to the combination and utilizing an alternative humectant in the combination described herein, the density of the solution is typically around 0.8 cm³-0.875 cm³.

Polyglyceryl-4 laurate/sebactate is known by those with skill in the art by multiple synonyms, such as dodecanoic acid, ABL, and lauric acid, and is alternatively commonly referred to as tetraglycerol. Polyglyceryl-4 laurate/sebacate and polyglyceryl-4 caprylate/caprate are used as emulsifiers. As is well known in the art, the addition of an emulsifier allows hydrophilic and hydrophobic molecules to disperse in each other, which creates a homogenous, smooth solution, and prevents separation of elements. Alternative emulsifiers may be used without going outside the scope intended. Some alternative emulsifiers are: esters of monoglycerides, esters of fatty acids, or lecithin. While there are other alternatives that may work just as effectively as an emulsifier, one embodiment contemplated specifically uses polyglyceryl-4 laurate/sebacate and/or polyglyceryl-4 caprylatercaprate as the emulsifiers.

Sodium polyacrylate is utilized in the present combination as a thickening agent in order to increase the viscosity of the liquid solution. The present embodiment utilizes sodium polyacrylate as a thickener, but there are certainly alternative thickeners which maybe used, inside the scope of the present disclosure. Some alternative thickening agents contemplated are carboxymethyl cellulose, ethylene maleic anhydride, polyethylene glycol, vegetable gums, petroleum jelly, and waxes or the like.

Moisturizers, also commonly referred to as emollients, are used to increase the water content of a subject's skin by slowing and reducing evaporation. The increased water content prevents dry or oily residue on the skin's outer layer. One example of a moisturizer that may be used is squalane. Squalane may also be known or listed as perhydrosqualene or dodecahydrosqualene. An isomer of squalene—such as isosqualane may additionally be added to the combination to enhance the moisturizing effects. There are many alternative moisturizers that are contemplated in the present disclosure having similar properties to squalane and thus being an alternative moisturizer: lightweight oils such as olive oil, castor oil, coconut oil, in(?) butters, avocados, petrolatum, paraffin waxes, and/or lanolin.

Aloe barbadensis loaf juice is also known by numerous synonyms, such as aloe vera, true aloe, aloe flava, and aloe indica, among other names. There are numerous uses for the aloe plant, such as protection from ultra-violet (UV) damage, as an anti-inflammatory, as a topical cream, as a scar reducer, for burn healing, as a humectant, as a moisturizer, and possibly for wound healing. In the present embodiment, the addition of aloe is used (among other things) to remove dead skin cells and toxins.

The composition further includes benzenesulfonic acid or salts thereof. The purpose of utilizing benzenesulfonic acid or salts thereof is as a processing aid and to enhance the tattoo under black light. As used herein, a processing aid is defined generally as a substance that is added to a compound or solution for its technical and functional effects in the processing of the compound or solution, but any presence in the finished compound or solution is insignificant, and does not have substantial technical or functional effect in the finished compound or solution. Alternatively, a salt of benzenesulfonic acid may be used, such as a besilate, or besylate, or a sulfonation using concentrated sulfuric acid, such as a sulfanilic acid, a toluenesulfonic acid, a besylic acid, or a phenylsulfonic acid, and any or all salts thereof.

Additionally, panthenol is added to the present combination, and is used as both a moisturizer and as a humectant. Panthenol has an additional effect of reducing itching and scabbing. Panthenol is also known by its trade name of Bepathen®. While the preferred humectant is panthenol, alternative embodiments may use similar humectants such as lecithin, glycerin, glycol, oleate, or vitamin B5.

In the preferred embodiment, a vitamin E supplement is used in the composition. Vitamin E is used herein as a generic term for a tocol and/or tocotrienol derivative. Vitamin E is typically used for moisturizing properties, or for its antioxidant properties. Specifically, dl-alpha-tocopherol, also known as dl-alpha-tocopheryl, is a synthetic vitamin E supplement. While any vitamin E supplement may be used in the current embodiment, dl-alpha-tocopheryl is preferred. The term “dl” as used, denotes that the product is a synthetic base, and alternatively, the prefix “d” may be used to indicate that the product was derived from a natural source. Some alternative names for similar vitamin E supplements that may be used are within a wide variety of the spectrum, such as tocopherol linoleate, acetate, or nicotinate. Generally, vitamin E exists in eight chemical forms: alpha-tocopherol, beta-tocopherol, gamma-tocopherol, and delta-tocopherol, as well as alpha-tocotrienol, beta-tocotrienol, gamma-tocotrienol, and delta-tocotrienol.

Another element of the combination is cold pressed biodegradable oil. Cold pressed extraction involves crushing the seed or nut to force out the, oil. Optionally, the seeds could be ground and crushed by a rotating screw as well. Application of heat will damage the oil which is why the process is termed cold pressed. A common cold pressed biodegradable oil used is vegetable oil. However, vegetable oil may take on a variety of types of plant oils, such as: coconut, corn, cottonseed, olive, palm, peanut, rapeseed, safflower, sesame, soybean, nut, sunflower, citrus, gourd, squash, watermelon, cocoa butter, avocado, apricot, grape seed, carrot, pomegranate, cranberry, hemp, kapok, kenaf, marula, mustard, quinoa, tobacco, tomato or pumpkin oil, among a wide variety of other vegetable oils. Although there are a wide variety of vegetable oils, they all perform the essential purpose desired for the present embodiment, which is to slow the loss of water from the subject's skin.

In the present embodiment, the combination includes a high alkaline hydrocarbon such as C-30 hydrocarbons. C-30, or C₃₀, as is, used and referred to herein, refers to the presence of 30 carbon atoms in the alkane, and is one of many different types of high alkaline hydrocarbons. C-30 is also referred to as triacontane, and this higher alkane hydrocarbon is utilized primarily for softening the skin. Other high alkaline hydrocarbons within the range of C₁₇-C₃₅ have similar properties, such as C₂₅ paraffin wax, but C₃₀ is the preferred hydrocarbon for the present embodiment.

In an alternative embodiment, phenoxyethanol is used as a preservative. A preservative, in either natural or synthetic form, is used to enhance the safety and is used to increase shelf stability of products. Many skilled in the art as well as those in parallel arts are aware that phenoxyethanol is known by a wide variety of pseudonyms, such as hydroxyethyl phenyl ether and phenoxy-ethanol, tirclosan, emery, sorbic acid, phenyl cellosolve as well as rose ether among others. The addition of phenoxyethanol is meant to preserve the length of time that the solution may be stored, also known as the shelf life. Additionally, phenoxyethanol may be added to prevent bacteria, mold, and/or fungi. There are alternative preservatives which may be used for similar effect, but phenoxyethanol is the preferred preservative contemplated. Some of the alternatives contemplated in other embodiments include propyl gallate, sulfites, paraben, or quaternium-15.

In one embodiment the formulation impregnating the towelette 12 may be compounded by the following materials listed in order of weight;

TABLE II

Water
White Mineral Oil
Glycerin
Phenoxyethanol
Polyglyceryl-4caprate/Carylate
Polyglyceryl-4 Laurate/Sebacate
Squalane
Sodium Polyacrylate
Isosqualane
Blend Of Cold Pressed Carrot Seed Oil
Pomegranate Seed Oil
Cranberry Seed Oil
Grape Seed Oil
Black Cumin Seed Oil
Panthenol
Dl-Alpha-Tocopheryl Acetate
Lodopropnyl Butycarbamate
C30 Hydrocarbons
Aloe Barbadensis Leaf Juice
Vitamin E

In another alternative embodiment, the combination described herein additionally includes a sunscreen. As described herein, sunscreen is used to mean a cream, lotion, spray, gel, or topical application that absorbs and/or reflects UV rays and UV radiation to protect against sunburn. The protection against UV rays and UV radiation will help to keep tattoo inks from lading. Additionally or alternatively, a melanocyte inhibitor may be used for similar effect. A melanocyte is a melanin-producing cell, and melanin is the pigment responsible for skin color, and for skin tanning. An inhibitor of the melanocyte thus inhibits the production of melanin, and prevents the skin from tanning. By keeping the skin lighter, any blemish, such as tattoo ink, has greater contrast, and is therefore more easily visible and vibrant.

In an alternative embodiment of the present disclosure, an exfoliator may be added to the combination to expedite the removal of dead skin cells. Dead skin cells can cause the skin to look rough, and dull. As is known, exfoliation is the removal of the dead, skin cells, and may he achieved through physical or chemical methods. The physical method typically involves using an abrasive surface, such as pumice, on the skin. Chemically, there are different forms of acids, such as alpha hydroxy acids or beta hydroxy acids that can be used, or an option is to use a glycolic acid, salicylic acid, or hyaluronic: acid mixture, or enzymes.

Another alternative embodiment is to utilize optical brighteners, such as optical brightening agents, fluorescent brightening agents, or fluorescent whitening agents. Typically, these are added, to enhance the appearance of color on the visible electromagnetic spectrum, such as with stilbenes, polyols, ear umbelliferone. The addition of any of these brighteners is for the purpose of brightening the appearance of the tattoo ink.

A towelette, as used herein, refers to a general fabric. The general fabric maybe disposable, but does not need to be as it could be washed and re-used. The fabric may consist of paper, cloth, wool, cotton, silk, and/or non-woven fibers. The material of the towelette is tangential to the purpose of the towelette, as the perceived intention is to be used as an applicator of the solution disclosed. Therefore, embodiments of the towelette may be offered in a wide variety of sizes while adhering to the spirit of the disclosure. The towelette contemplated herein may be reusable after a wash, but preferably is a single use towelette. If an embodiment of the towelette is single use, the towelette may be dispensed in a single foil pack, a soft pack, or a canister, among other methods. The purpose of the dispensing method of being sealed is to prevent the towelette and solution located therein from exposure to the air and atmosphere.

In still another aspect, a method of applying the combination to enhance and/or moisturize a tattoo is described, with the method comprising moistening a towelette as described before with an embodiment of the combination discussed above. The skin portion of said tattoo of the subject is, exposed, and the towelette is pressed against said tattoo, so that the combination is applied to the skin. The towelette is then oscillated against the skin, which is in a back-and-forth motion, or perhaps in a generally circular motion. The oscillating motion may be in any pattern without deviating from the scope of the present disclosure.

Preferably, the material used to moisten the towelettes is free from solvents that could affect tattoo pigments. Alternative embodiments of the invention contemplate that a sunscreen, a melanocyte inhibitor and an exfoliator maybe added. In addition or alternatively, other optical brightening agents (OBAs), fluorescent brightening agents (FBAs) or fluorescent whitening agents (FWAs) can be used to absorb light in the ultraviolet and violet region (usually 340-370 nm) of the electromagnetic spectrum, and re-emit light in the blue region (typically 420-470 nm). The most common classes of compounds with this property are the stilbenes, e.g., 4,4′-diamino-2,2′-stilbenedisulfonic acid.

The towelettes are nonwoven material that can vary in size. The towelettes can be dispensed in a single foil pack, canister, or soft pack. In use, the towelettes can be removed from a foil packet or from a canister that may be sealed in between uses and applied to the area of the skin with the tattoo with a wiping motion, for example, in a generally circular pattern. It is not necessary to treat the skin outside of the area of the tattoo. The materials used to pre-moisten the towelette enhance the optical properties of the skin to bring out greater contrast and saturation in the tattoo art when that material is applied to the skin.

In another embodiment contemplated, the combination of elements disclosed consists of multiple elements comprising a solution. The elements are a combination of: water, mineral oil, glycerol, polyglyceryl -4 laurate/sebactate, polyglyceryl-4 caprylate/caprate, phenoxyethanol IPBC, sodium polyacrylate, squalene, isosqualane, aloe barbadensis leaf juice, benzenesulfonic acid or salts thereof, panthenol, dl-alpha-tocopheryl acetate, cold pressed biodegadable oils, and c-30 hydrocarbons. The combination may be an assortment of these elements, and does not need to be all of the elements to embody the present concept.

Certain terminology is used herein for purposes of reference only, and thus is not intended to be limiting. For example, terms such as “upper”, “lower”, “above”, and “below” refer to directions in the drawings to which reference is made. Terms such as “front”, “back”, “rear”, “bottom” and “side”, describe the orientation of portions of the component within a consistent but arbitrary frame of reference which is made clear by reference to the text and the associated drawings describing the component under discussion. Such terminology may include the words specifically mentioned above, derivatives thereof, and words of similar import. Similarly, the terms “first”, “second” and other such numerical terms referring to structures do not imply a sequence or order unless clearly indicated by the context.

When introducing elements or features of the present disclosure and the exemplary embodiments, the articles “a”, “an”, “the” and “said” are intended to mean that there are one or more of such elements or features. The terms “comprising”, “including” and “having” arc intended to be inclusive and mean that there may be additional elements or features other than those specifically noted. It is further to be understood that the method steps, processes, and operations described herein are not to be construed as necessarily requiring their performance in the particular order discussed or illustrated, unless specifically identified as an order of performance. It is also to be understood that additional or alternative steps may be employed.

It is specifically intended that the present invention not be limited to the embodiments and illustrations contained herein and the claims should be understood to include modified forms of those embodiments including portions of the embodiments and combinations of elements of different embodiments as come within the scope of the following claims. All of the publications described herein, including patents and non-patent publications are hereby incorporated herein by reference in their entireties.

What is claimed is:

1. A tattoo-enhancing wipe comprising a towel impregnated with a tattoo color enhancing oil.

2. The tattoo-enhancing wipe of claim 1 further including an emulsifier and water.

3. The tattoo-enhancing wipe of claim 2 wherein an amount of water exceeds the amount of enhancing oil by weight.

4. The tattoo-enhancing wipe of claim 3 further including a humectant.

5. The tattoo-enhancing wipe of claim 4 wherein the oil is a mineral oil.

6. The tattoo-enhancing wipe of claim 1 further including a superabsorbent polymer.
